(19) United States
(12) Patent Application Publication
(10) Pub. No.: US 2015/0252429 A1
(43) Pub. Date: Sep. 10, 2015

(54) DETECTION OF BRAIN CANCER TYPES

(71) Applicants: Nathan D. PRICE, Seattle, WA (US); Leroy HOOD, Seattle, WA (US); Jaeyun SUNG, Seattle, WA (US); Donald GEMAN, Seattle, WA (US); INSTITUTE FOR SYSTEMS BIOLOGY, Seattle, WA (US)

(72) Inventors: Nathan D. Price, Seattle, WA (US); Leroy Hood, Seattle, WA (US); Jaeyun Sung, Gwangju (KR); Donald Geman, Baltimore, MD (US)

(21) Appl. No.: 14/439,974

(22) PCT Filed: Oct. 31, 2013
(86) PCT No.: PCT/US2013/067890

§ 371 Date: Apr. 30, 2015

Related U.S. Application Data
(60) Provisional application No. 61/720,947, filed on Oct. 31, 2012.

(51) Int. Cl. C12Q 1/68

(52) U.S. Cl. CPC C12Q 1/6886; C12Q 2600/112; C12Q 2600/16; C12Q 2600/158

(57) ABSTRACT

The invention provides methods to identify various types of brain cancer tissue by comparing gene expression transcriptomes in tissue samples. A sequential method to discriminate among six different types of brain cancer is described. The invention relates to the field of markers for various types of brain cancer. More particularly, it relies on a sequential system for sorting individual cancer types.

### STATEMENT OF RIGHTS TO INVENTIONS MADE UNDER FEDERALLY SPONSORED RESEARCH

This invention was supported in part by a National Institutes of Health/National Center for Research Resources Grant UL1 RR 025005 (DG), and the Grand Duchy of Luxembourg-Institute for Systems Biology Program (LH, NDP). The U.S. government has certain rights in this invention.

### TECHNICAL FIELD

The invention relates to the field of markers for various types of brain cancer. More particularly, it relies on a sequential system for sorting individual cancer types.

### BACKGROUND ART

Identification markers for various types of disease conditions have been developed based on gene expression data. Assessment of the transcriptome has been able to identify various markers for diagnosis, prognosis prediction and optimal therapy of various cancers (Friedman, D. R., et al., Clin. Cancer Res. (2009) 15:6947-6955; Khan, J., et al., Nature Med. (2001) 7:673-679; Yeoh, E. J., et al., Cancer Cell (2002) 1:133-143).

These studies, while useful, exhibit a wide variation among various datasets obtained for particular types of cancer. These disparate results may be accounted for by differing methodologies, different demographics among the subjects, individual variation in cancer heterogeneity, and, perhaps, different measurement techniques. Meta-analyses that compile a multiplicity of studies as a basis for judgment have, to some extent, alleviated the problems caused by this variability (Miller, J. A., et al., PNAS (2010) 107:12698-12703; Dudley, J. T., et al., Molecular Systems Biol. (2009) 5:307). However, such meta-analysis has not been provided with respect to determination of markers for various brain cancers.

In addition, others have experimented with data-driven hierarchical approaches to multi-category classification in the context of machine learning (Blanchard, G., et al., Am. Stat. (2005) 33:1155-11202; Amit, Y., et al., IEEE Transactions on Pattern Analysis and Machine Intelligence (2004) 26:1606-1621).

The present inventors have marshaled these techniques specifically with respect to determination and verification of successful gene expression markers for various types of brain tumors.

### DISCLOSURE OF THE INVENTION

The invention provides a panel that successfully can distinguish cancerous brain tissue from normal brain tissue, and further can distinguish among six different types of brain cancer with high levels of sensitivity and specificity in correlation with phenotypic assessments. The panel can be employed in a hierarchical discrimination sequence to parse tissues into these six cancerous types. It employs a framework for brain cancer diagnosis that is a tree-structured hierarchy of these brain cancer phenotypes.

Thus, in one aspect, the invention is directed to a panel for distinguishing among normal brain tissue, samples that harbor meningioma (MNG), samples that harbor ependymoma (EPN), samples that harbor medulloblastoma (MDL), samples that harbor glioblastoma (GBM), samples that harbor oligodendroglioma (OLG), and samples that harbor pilocytic astrocytoma (PA) wherein said panel comprises detection reagents for the transcripts of the following genes: PRPF40A and PURA; NRCAM and ISLR; IDH2 and GMDS; SALL1 and PAFAH1B3; SRI and NBEA; DDR1 and TIA1 or MAB21L1; ITPKB and PDS5B; NUP62CL and ZNF280A; GALNS and WAS; CELSR1 and OR10H3; TLE4 and OLIG2; DDX27 and KCNMA1; COX7A2 and GNPTAB; GNPTAB and NDUFS2; APOD and PPIA; CD59 and SNRPB2; SEMA3E and ADAMTS3; HINT1 and CD59; BAMBI and CIAPIN1; FLNA and TNKS2; ITGB3BP and RB1CC1; DDX27 and TRIM8; and LARP5 and ANXA1.

In another aspect, the invention is directed to a method to distinguish among normal brain tissue, samples that harbor MNG, samples that harbor EPN, samples that harbor MDL, samples that harbor GBM, samples that harbor OLG, and samples that harbor PA which method comprises initially distinguishing normal brain tissue from tissue with all of the above-mentioned MNG, EPN, MDL, GBM, OLG and PA, followed by distinguishing samples that harbor MNG from samples that harbor EPN, MDL, GBM, OLG or PA, followed by distinguishing samples that harbor MDL from samples that harbor EPN, GBM, OLG or PA, followed by distinguishing samples that harbor EPN from samples that harbor GBM, OLG or PA, followed by distinguishing samples that harbor PA from samples that harbor GBM or OLG, followed by distinguishing between samples that harbor GBM and samples that harbor OLG.

The invention is thus directed to methods to distinguish individual types of cancers in the context of this method and to kits for performing various portions of the method.

In still another aspect, the invention is directed to a method to identify brain cancer or other disease markers by meta-analysis of multiple datasets designed to identify such markers.

### BRIEF DESCRIPTION OF THE DRAWINGS

FIG. 1A shows a diagrammatic representation of the hierarchical method of the invention. FIG. 1B is a further diagrammatic description of the method.

FIGS. 2A-2F compare various methods of integrating multiple datasets.

### MODES OF CARRYING OUT THE INVENTION

The invention takes advantage of the results from multiple datasets and applies a specific algorithm to order the markers derived from these datasets into a hierarchical system for discriminating between normal tissue and among six different types of brain cancers.

Data-driven, hierarchical approaches to multi-category classification have been investigated extensively in machine learning. A classification framework in the form of a tree-structured hierarchy of sets of different categories, is first designed followed by identifying binary classifiers for all decision points (i.e., nodes and/or edges) of the tree. The sets of binary classifiers are aggregated into a classifier marker-panel, which directs diagnosis of a sample from a subject down the hierarchical structure towards a particular phenotype. The cumulative expression patterns constitute “hierarchically-structured” diagnostic signatures.

A computational approach called Identification of Structured Signatures And Classifiers (ISSAC) based on this idea was developed to identify diagnostic signatures that simultaneously distinguishes major cancers of the human brain. From an integrated dataset of publicly available gene expression data, ISSAC provided a global diagnostic hierarchy and corresponding brain cancer signatures composed of sets of gene-pair classifiers. Integration of datasets from multiple studies enhances the disease signal sufficiently to mitigate batch effects and improve independent validation results.

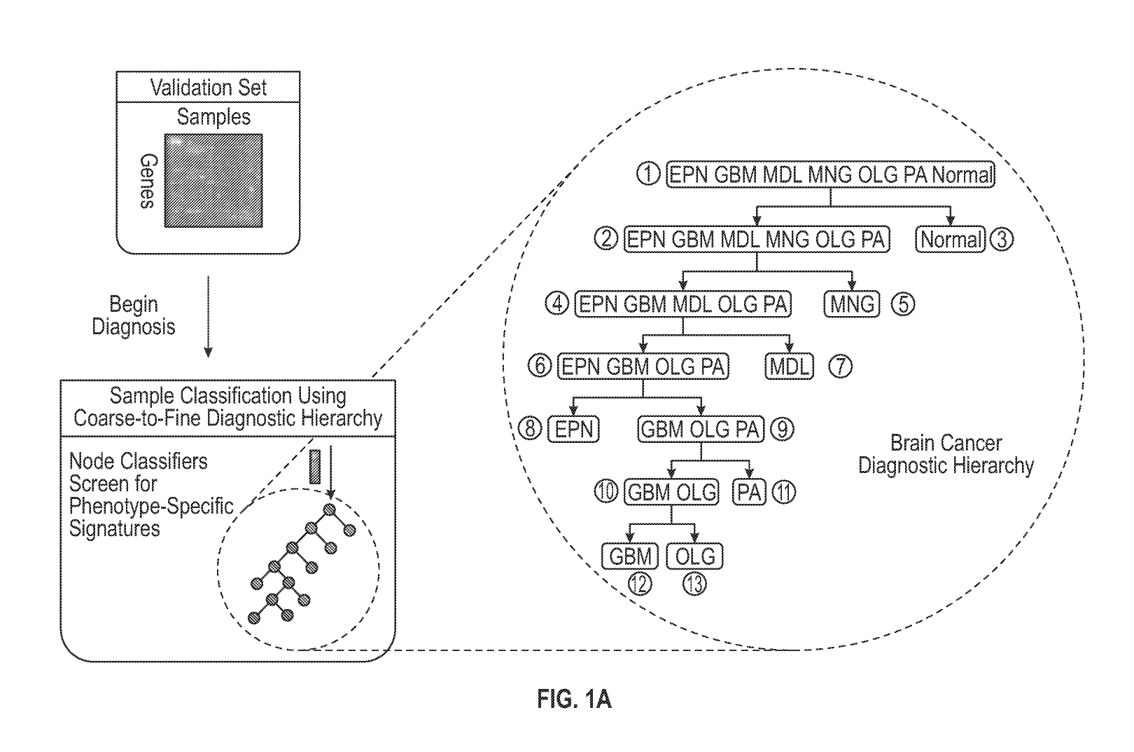

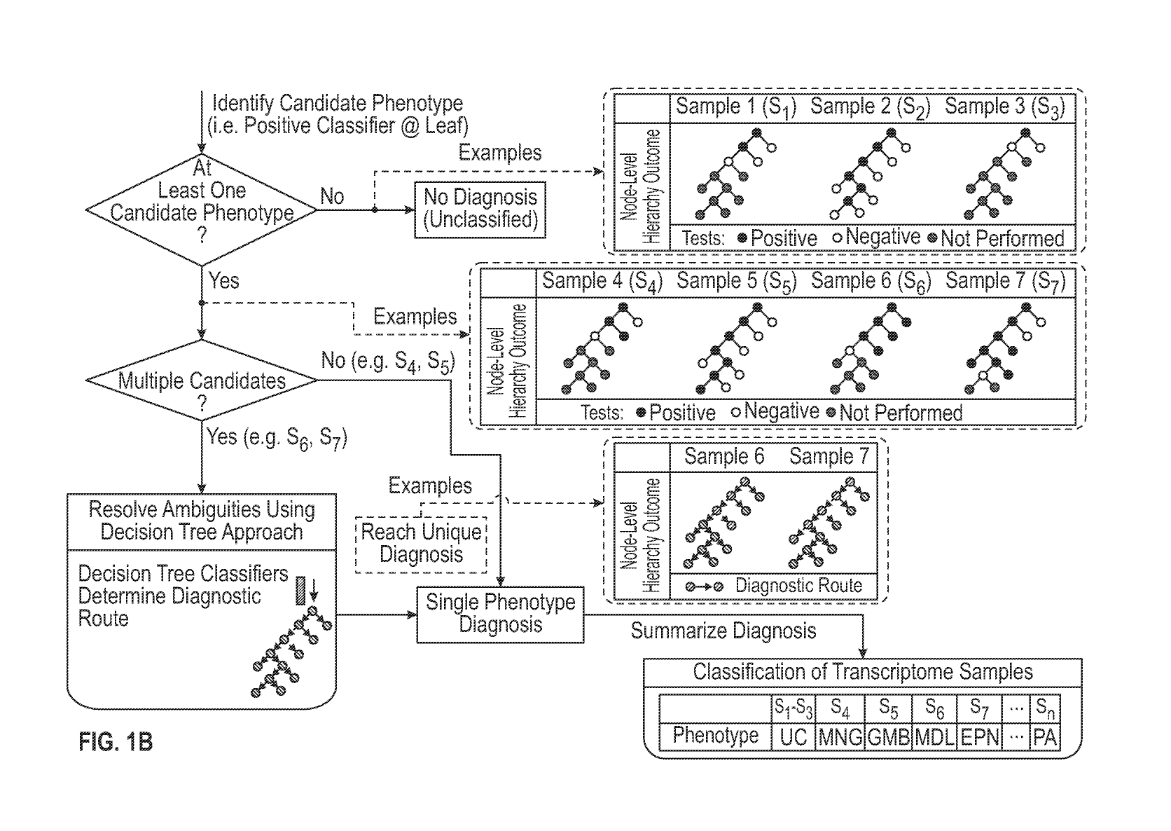

ISSAC constructs the framework for brain cancer diagnosis as shown in FIG. 1A—a tree-structured hierarchy of all brain cancer phenotypes built using an agglomerative hierarchical clustering algorithm on gene expression training data. Briefly, the construction of the hierarchy relies on the fact that there exist natural groupings among phenotypes based on shared features in their gene expression. As the set of different phenotypes is partitioned into smaller and more homogeneous subsets, the multi-class diagnosis problem is thereby decomposed into more tractable sub-problems.

FIG. 1A shows comprehensive classification of human brain cancer and normal brain transcriptomes using diagnostic signatures from ISSAC. As shown, the coarse-to-fine classification process is represented by a hierarchical structure of phenotype groupings. The diagnostic hierarchy has thirteen nodes in total, and seven terminal nodes (i.e., leaves). The node classifiers are executed sequentially and adaptively on a given expression profile; a classifier test for a particular node is performed if and only if all of its ancestor tests were performed and deemed positive. The node classifiers are used to screen for phenotype-specific signatures.

As shown in FIG. 1B, leaves that have positive classifier outcomes correspond to the candidate phenotypes of a given expression profile. If there is no candidate phenotype, the expression profile is labeled as ‘Unclassified’. If only one candidate phenotype is identified, the profile is labeled as the phenotype of the respective leaf. If the profile is considered to consist of multiple phenotype signatures, the ambiguity is resolved using the decision-tree classifiers based on the same diagnostic hierarchy. Here, the decision-tree classifiers are executed starting from the root of the tree, directing the profile to one of the two child nodes sequentially until it completes a full path towards a leaf The phenotype label of the final destination corresponds to the unique diagnosis.

ISSAC identifies a binary classifier corresponding to each node and to each edge of the diagnostic hierarchy. Briefly, each classifier attempts to distinguish between two sets of phenotypes. These classifiers are based on comparing the relative expression values (i.e., ranks) between two genes, or for one or several pairs of genes within a gene expression profile at each stage. The chosen pairs are the ones that best differentiate between the phenotype sets, and are based entirely on the reversal of relative expression, as previously reported (Geman, D., Stat. Apps. in Gen. & Mol. Biol. (2004) 3:Article 19. Briefly, the decision rule by Geman, et al. consists of two genes (gene i and gene j), distinguishing two phenotypes (class A and class B): If the expression of gene i is greater than that of gene j for a given profile, then the phenotype is classified as class A; otherwise, class B. Recently, it has been shown that using such simple decision rules with only a small number of gene-pairs can lead to highly accurate supervised classification of human cancers (Tan, A. C., et al., Bioinformatics (2005) 21:3896-3904).

The objective of a node classifier is to distinguish the set of phenotypes associated with the node from all other phenotypes. Overall, the node classifiers represent a series of coarse-grained to fine-grained explanations of the hierarchical groupings, and are used in diagnosis to screen for phenotype-specific expression patterns. Thus, the hierarchy of binary predictors guides classification of an expression profile in a dynamic “coarse-to-fine” fashion: a classifier is executed if and only if all of its ancestor classifiers have been executed and returned a positive response, i.e., predicted the phenotypes in each node. The cumulative outcome of the node classifiers for a given expression profile is the set of its candidate phenotypes, corresponding to all the leaves of the hierarchy that were reached successfully.

For tie-breaking purposes, ISSAC also identifies classifiers at the edges of the diagnostic hierarchy. The objective of these classifiers is analogous to that of decision rules of an ordinary decision-tree: to distinguish the two sets of phenotypes associated with the two child nodes. The cumulative outcome of the decision-tree classifiers is a unique diagnosis.

Step-By-Step Description of How ISSAC Works

Construction of the Disease Diagnostic Hierarchy

Let £=(d₁, . . . , d₇) be the collection of class labels, where dᵢ denotes brain phenotype i. Using expression profiles of the phenotype classes, we first calculate the Top Scoring Pair (TSP) score (Δ) of all gene-pair combinations between all pair-wise class comparisons. As previously described (17), the TSP score between two classes dₘ and dₙ, of two genes, gene i and gene j, is defined as:

Δ_{i,j}(dₘ,dₙ)=|Pi>j(dₘ)−Pi>j(dₙ)|,

where Pi>j(dₘ) and Pi>j(dₙ) denotes the percentage of samples in dₘ and dₙ, respectively, whose expression of gene i is higher than that of gene j. Δₘₐₓ(dₘ, dₙ) denotes the maximum Δ_{i,j} between dₘ and dₙ over all gene pairs i and j.

Let C designate an evolving set of groups of labels that starts off as the set of individual class (d₁, . . . , d₇). The brain disease diagnostic hierarchy was constructed by progressively evolving C towards the set of all groupings in the hierarchy using the following steps:

1. For all pair-wise comparisons of distinct elements in C, we calculate all Δₘₐₓ. The leaves of the class-pair dₘ and dₙ with the smallest value of Δₘₐₓ are merged into the first node of the tree, denoted as n_{d}_{ₘ}_{,d}_{ₙ}.

2. Δₘₐₓ of all pair-wise comparisons of the elements in the updated C are calculated, and the pair with the smallest value of Δₘₐₓ is grouped into the next node of the tree. Since at this point C contains one non-singleton node and a host of other leaves, the next merging can be either between two leaves dᵤ and dᵥ, denoted as n_{d}_{ᵤ}_{,d}_{ᵥ}, or between a node n_{d}_{ₘ}_{,d}_{ₙ} and a leaf dᵤ, denoted as n_{d}_{ₘ}_{,d}_{ₙ}_{d}_{ᵤ}. Whichever pair with the smallest Δₘₐₓ merges to form a new node in C.

3. This process of finding the minimum Δₘₐₓ for all pair-wise elements in C, and adding the new node in C, is iterated until all nodes and leaves are connected to form a tree structure. All classes combine to form the top node n_{d}_{₁}_{, . . . , d}_{₇} at the top of the diagnostic hierarchy (i.e., root).

The Markers Used in the Invention Method:

The classifier transcriptome gene expression markers are shown in Table 1.

TABLE 1

| | | Gene i^{c} | Gene j^{c} | |
|---|---|---|---|---|
| Node #^{a} | Node classes^{b} | Gene symbols | Gene symbols | k^{d} |
| 2 | EPN GBM MDL | PRPF40A | PURA | 1 |
| | MNG OLG PA | | | |
| 3 | normal | PURA | PRPF40A | 1 |
| 4 | EPN GBM MDL | NRCAM | ISLR | 1 |
| | OLG PA | IDH2 | GMDS | |
| 5 | MNG | ISLR | NRCAM | 1 |
| 6 | EPN GBM | SALL1 | PAFAH1B3 | 2 |
| | OLG PA | SRI | NBEA | |
| | | DDR1^{e} | TIA1 | |
| | | DDR1^{e} | MAB21L1 | |
| | | ITPKB | PDS5B | |
| 7 | MDL | PAFAH1B3 | SALL1 | 4 |
| | | NBEA | SRI | |
| | | TIA1 | DDR1^{e} | |
| | | MAB21L1 | DDR1^{e} | |
| | | PDS5B | ITPKB | |
| 8 | EPN | NUP62CL | ZNF280A | 2 |
| | | GALNS | WAS | |
| | | CELSR1 | OR10H3 | |
| | | TLE4 | OLIG2 | |
| 9 | GBM OLG PA | ZNF280A | NUP62CL | 1 |
| 10 | GBM OLG | DDX27 | KCNMA1 | 1 |
| | | COX7A2 | GNPTAB | |
| 11 | PA | KCNMA1 | DDX27 | 3 |
| | | GNPTAB | NDUFS2 | |
| | | APOD | PPIA | |
| | | CD59 | SNRPB2 | |
| | | SEMA3E | ADAMTS3 | |
| | | CD59 | HINT1 | |
| | | BAMBI | CIAPIN1 | |
| 12 | GBM | FLNA | TNKS2 | 1 |
| | | ITGB3BP | RB1CC1 | |
| | | DDX27 | TRIM8 | |
| 13 | OLG | LARP5 | ANXA1 | 1 |

Thus, the marker panels consist of 39 total gene pairs and 44 unique genes. The 44 genes are available as a subset of Affymetrix® microarrays.

In this table, ^{a}Node # corresponds to numerical labels in the diagnostic hierarchy shown in FIG. 1. ^{b}Disease abbreviation (name): EPN (Ependymoma), GBM (Glioblastoma Multiforme), MDL (Medulloblastoma), MNG (Meningioma), OLG (Oligodendroglioma), PA (Pilocytic astrocytoma), and normal (Normal brain). ^{c}Gene i and gene j are the genes expressed higher and lower, respectively, within each gene-pair classification decision rule. Specifically, the statement of “Gene i is expressed higher than Gene j” being true contributes to the expression profile being classified as the phenotype(s) of the node. Gene names, chromosome loci, and Affymetrix® microarray platform probe IDs of the classifier genes are in Table 2 below. ^{d}The minimum number of gene-pair classifiers whose decision rule outcomes for an expression profile are required to be ‘true (=1)’ for the profile to be classified as the phenotype(s) of the node. ^{e}Genes that share same symbol/name, but correspond to different Affymetrix® probe IDs.

TABLE 2
Node marker-panel for brain cancer and normal transcriptome classification

| | Node | Gene i | | | | Gene j | | | | |
|---|---|---|---|---|---|---|---|---|---|---|
| Node | phenotype | | | Chromosome | Affymatrix | | | Chromosome | Affymatrix | |
| # | classes | Gene symbol | Gene name | locus | Probe ID | Gene symbol | Gene name | locus | Probe ID | k |
| 2 | EPN | PRPF40A | PRP40 pre-mRNA processing factor 40 | 2q23.3 | 218053_at | PURA | Purine-rich element binding protein A | 5q31 | 204021_s_at | 1 |
| | GBM | | homolog A (S. cerevisiae) | | | | | | | |
| | MDL | | | | | | | | | |
| | MNG | | | | | | | | | |
| | OLG PA | | | | | | | | | |
| 3 | normal | PURA | Purine-rich element binding protein A | 5q31 | 204021_a_at | PRPF40A | PRP40 pre-mRNA processing factor 40 | 2q23.3 | 218053_at | 1 |
| | | | | | | | homolog A (S. cerevisiae) | | | |
| 4 | EPN | NRCAM | Neuronal cell adhesion molecule | 7q31 | 204105_a_at | ISLR | Immunoglobulin superfamily containing | 15q23-q24 | 207191_s_at | 1 |
| | GBM | | | | | | leucine-rich repeat | | | |
| | MDL | | | | | | | | | |
| | OLG PA | | | | | | | | | |
| | | IDH2 | Isocitrate dehydrogenase 2 (NADP+), | 15q26.1 | 210046_s_at | GMDS | GDP-mannose 4,6-dehydratase | 6p25 | 214106_s_at | |
| | | | mitochondrial | | | | | | | |
| 5 | MNG | ISLR | Immunoglobulin superfamily containing | 15q23-q24 | 207191_s_at | NRCAM | Neuronal cell adhesion molecule | 7q31 | 204105_s_at | 1 |
| | | | leucine-rich repeat | | | | | | | |
| 6 | EPN | SALL1 | Sal-like 1 (Drosophila) | 16q12.1 | 206893_at | PAFAH1B3 | Platelet-activating factor acetylhydrolase 1b, | 19q13.1 | 203226_at | 2 |
| | GBM | | | | | | catalytic subunit 3 | | | |
| | OLG PA | SRI | Sorcin | 7q21 | 208920_at | NBEA | Neurobeachin | 13q13 | 221207_s_at | |
| | | DDR1 | Discoidin domain receptor tyrosine kinase 1 | 6p21.3 | 210749_x_at | TIA1 | TIA1 cytotoxic granule-associated RNA | 2p13 | 201447_at | |
| | | | | | | | binding protein | | | |
| | | DDR1 | Discoidin domain receptor tyrosine kinase 1 | 6p21.3 | 208779_x_at | MAB21L1 | Mab-21-like 1 (C. elegans) | 13q13 | 206163_at | |
| | | ITPKB | Inositol 1,4,5-trisphosphate 3-kinase B | 1q42.13 | 203723_at | PDS5B | PDS5, regulator of cohesion maintenance, | 13q12.3 | 204742_s_at | |
| | | | | | | | homolog B (S. cerevisiae | | | |
| 7 | MDL | PAFAH1B3 | Platelet-activating factor acetylhydrolase 1b, | 19q13.1 | 203228_at | SALL1 | Sal-like 1 (Drosophila) | 18q12.1 | 206893_at | 4 |
| | | | catalytic subunit 3 | | | | | | | |
| | | NBEA | Neurobeachin | 13q13 | 221207_s_at | SR1 | Sorcin | 7q21 | 208920_at | |
| | | TIA1 | TIA1 cytotoxic granule-associated RNA binding | 2p13 | 201447_at | DDR1 | Discoidin domain receptor tyrosine kinase 1 | 6p21.3 | 210749_x_at | |
| | | | protein | | | | | | | |
| | | MAB21L1 | Mab-21-like 1 (C. elegans) | 13q13 | 206163_at | DDR1 | Discoidin domain receptor tyrosine kinase 1 | 6p21.3 | 208779_x_at | |
| | | PDS5B | PDS5, regulator of cohesion maintenance, | 13q12.3 | 204742_s_at | ITPKB | Inositol 1,4,5-trisphosphate 3-kinase B | 1q42.13 | 203723_at | |
| | | | homolog B (S. cerevisiae | | | | | | | |
| 8 | EPH | NUP62CL | Nucleoporin 62 kDa C-terminal like | Xq22.3 | 220520_s_at | ZNF280A | Zinc finger protein 280A | 22q11.22 | 216034_at | 2 |
| | | GALNS | Galactosamine (N-acetyl)-6-sulfate sulfatase | 16q24.3 | 206335_at | WAS | Wiskott-Aldrich syndrome (eczema- | Xp11.4-p11.21 | 38964_r_at | |
| | | | | | | | thrombocytopenia) | | | |
| | | CELSR1 | Cadherin, EGF LAG seven-pass G-type | 22q13.3 | 41660_at | OR10H3 | Olfactory receptor, family 10, subfamily H, | 13p13.1 | 208520_at | |
| | | | receptor 1 (flamingo homolog, Drosophila) | | | | member 3 | | | |
| | | TLE4 | Transducin-like enhancer of split 4 (E(sp1) | 9q21.31 | 216997_x_at | OLIG2 | Oligodendrocyte lineage transcription factor 2 | 21q22.11 | 213824_at | |
| | | | homolog, Drosophila) | | | | | | | |
| 9 | GBM | ZNF280A | Zinc finger protein 280A | 22q11.22 | 216034_at | NUP62CL | Nucleoporin 62 kDa C-terminal like | Xq22.3 | 220520_s_at | 1 |
| | OLG PA | | | | | | | | | |
| 10 | GBM | DDX27 | DEAD (Asp-Glu-Ala-Asp) box polypeptide 27 | 20q13.13 | 215693_x_at | KCNMA1 | Potassium large conductance calcium- | 10q22.3 | 221584_s_at | 1 |
| | OLG | | | | | | activated channel, subfamily M, alpha | | | |
| | | | | | | | member 1 | | | |
| | | COX7A2 | Cytochrome c oxidase subunit VIIa polypeptide 2 | 6q12 | 217249_x_at | GNPTAB | N-acetylglucosamine-1-phosphate transferase, | 12q23.2 | 212959_s_at | |
| | | | (liver) | | | | alpha and beta subunits | | | |
| 11 | PA | KCNMA1 | Potassium large conductance calcium-activated | 10q22.3 | 221584_s_at | DDX27 | DEAD (Asp-Glu-Ala-Asp) box polypeptide 27 | 20q13.13 | 215693_x_at | 3 |
| | | | channel, subfamily M, alpha member 1 | | | | | | | |
| | | GNPTAB | N-acetylglucosamine-1-phosphate transferase, | 12q23.2 | 212959_s_at | NDUFS2 | NADH dehydrogenase (ubiquinone) Fe—S | 1q23 | 201966_at | |
| | | | alpha and beta subunits | | | | protein 2, 49 kDa (NADH-coenzyme Q | | | |
| | | | | | | | reductase) | | | |
| | | APOD | Apolipoprotein D | 3q26.2-qter | 201525_at | PPIA | Peptidylprolyl isomerase A (cyclophilin A) | 7p13 | 211378_x_at | |
| | | CD59 | CD59 molecule, complement regulatory protein | 11p13 | 212463_at | SNRPB2 | Small nuclear ribonucleoprotein polypeptide B | 20p12.1 | 202505_at | |
| | | SEMA3E | Sema domain, immunoglobulin domain (Ig), short | 7q21.11 | 206941_x_at | ADAMTS3 | ADAM metallopeptidase with thrombospondin | 4q13.3 | 214913_at | |
| | | | basic domain, secreted, (semaphorin) 3E | | | | type 1 motif, 3 | | | |
| | | CD59 | CD59 molecule, complement regulatory protein | 11p13 | 200985_s_at | HINT1 | Histidine triad nucleotide binding protein 1 | 5q31.2 | 208826_x_at | |
| | | BAMBI | BMP and activin membrane-bound inhibitor | 10p12.13-p11.2 | 203304_at | CIAPIN1 | Cytokine induced apoptosis inhibitor 1 | 16q13-q21 | 208968_s_at | |
| | | | homolog (Xenopus laevis) | | | | | | | |
| 12 | GBM | FLNA | Filamin A, alpha | Xq28 | 214752_x_at | TNKS2 | Tankyrase, TRF1-interacting ankyrin-related | 10q23.3 | 218228_s_at | 1 |
| | | | | | | | ADP-ribose polymerase 2 | | | |
| | | ITGB3BP | Integrin beta 3 binding protein (beta3-endonexin) | 1p31.3 | 205176_s_at | RB1CC1 | RB1-inducible coiled-coil 1 | 8q11 | 202034_x_at | |
| | | DDX27 | DEAD (Asp-Glu-Ala-Asp) box polypeptide 27 | 20q13.13 | 215693_x_at | TRIM8 | Tripartite motif-containing 8 | 10q24.3 | 221012_s_at | |
| 13 | OLG | LARP5 | La ribonucleoprotein domain family, member 4B | 10p15.3 | 208953_at | ANXA1 | Annexin A1 | 9q12-q21.2 | 201012_at | 1 |

The notations in Table 2 are as follows:

Node #: Corresponds to numerical labels shown in the brain phenotype diagnostic hierarchy (FIG. 1A). Brain phenotype abbreviation (name): ALZ (Alzheimer's), GBM (Glioblastoma multiforme), MDL (Medulloblastoma), MNG (Meningioma), normal (Normal brain), OLG (Oligodendroglioma), and PA (Pilocytic astrocytoma). Gene i/Gene j: the gene expressed higher and lower in the gene-pair, respectively, within each corresponding phenotype. Gene name/Chromosome locus: according to Entrez Gene. Affymetrix® Probe ID: For both Affymetrix® Human Genome U133A and U133Plus2.0 Arrays. k: The minimum number of gene-pair classifiers whose decision rule outcomes for a test sample are required to be ‘true (=1)’ for the sample to be classified as the phenotype(s) of the corresponding node.

To distinguish normal brain tissue from the six cancer types, only a single gene pair need be analyzed—a higher expression of PRPF40A than PURA classifies the tissue as cancerous. In the next step, only a single pair is required to distinguish MNG from the remaining cancer types; a higher expression of ISLR compared to NRCAM classifies the tissue as MNG. On the other hand, to distinguish MDL from the four cancer types EPM, GBM, OLG or PA, it has been found that two pairs need to be compared.

ISSAC uses the gene-pair classifiers for class prediction as described above and shown in FIG. 1B. Briefly, given a gene expression profile, ISSAC executes the node classifiers in a hierarchical, top-down fashion within the disease diagnostic hierarchy to identify the phenotype(s) whose class-specific signature(s) is present. In case of multiple class candidates (i.e., node classifiers for multiple leaves are positive), the ambiguity is resolved, if desired, by aggregating all the decision-tree classifiers into a classification decision-tree, thereby leading any expression signature down one unique path toward a single phenotype. Overall, we generated a diagnostic marker-panel whose classifiers allow efficient brain cancer diagnosis and straightforward, biologically meaningful interpretation. FIG. 1B is essentially a flow chart of decisions made using the tree of FIG. 1A, including dealing with multiple positive diagnoses from initial results.

The following examples are intended to illustrate but not to limit the invention.

### EXAMPLE 1

### Multi-Study Dataset of Human Brain Cancer Transcriptomes

All transcriptomic data used in our analysis are publicly available at the NCBI Gene Expression Omnibus (GEO). We integrated 921 microarray samples of six brain cancers which are ependymoma (EPN), glioblastoma multiforme (GBM), medulloblastoma (MDL), meningioma (MNG), oligodendroglioma (OLG), pilocytic astrocytoma (PA) and normal brain across 16 independent studies into a transcriptome meta-dataset. Importantly, we obtained the raw data (.CEL files) from each of these studies and preprocessed them simultaneously using identical techniques to reduce extraneous sources of technical artifacts (discussed below). All data manipulation and numerical calculations were performed using MATLAB (MathWorks).

We used the following strict criteria and reasoning to select brain phenotypes, to ensure data quality, and to help control for systemic bias:

1. To facilitate data integration, expression profiles must have been conducted on either the Affymetrix® Human Genome U133A or U133 Plus 2.0 microarray platform. This allowed maximum microarray sample collection without considerable reduction in number of overlapping classifier features (i.e., microarray probe-sets).

2. Transcriptomic datasets (i.e., GSE xxx) for each phenotype must have been collected from at least two independent sources to help mitigate batch effects.

3. All datasets must have consisted of no fewer than 5 microarray samples.

4. All datasets must have originated from primary brain tumor or tissue biopsies. Expression profiles from cell-lines or laser micro-dissections were not used in our study to better ensure sample consistency.

5. Raw microarray intensity data (.CEL files) must have been available on GEO for consensus preprocessing.

6. Sample preparation protocol must have been fully disclosed on GEO.

7. All microarray samples in a dataset of a given phenotype were used in order to take into consideration all sources of heterogeneity.

After an exhaustive search on GEO, we were able to find 921 microarray samples from 16 studies that met the above criteria. Information on all datasets (e.g., publication sources, Affymetrix® platforms, GEO dataset IDs, and microarray sample IDs) used in Table 3 and Table 4.

TABLE 3
Description of all GEO microarray datasets used in this study*

| | GEO | First author | | Sample | |
|---|---|---|---|---|---|
| Phenotype name | accession # | (publication year) | Ref. | size | Affymetrix array |
| Ependymoma | GSE16155 | Donson (2009) | S1 | 19 | U133 plus2.0 |
| | GSE21687 | Johnson (2010) | S2 | 83 | U133 plus2.0 |
| Glioblastoma | GSE 4412 | Freije (2004) | S3 | 59 | U133A |
| Multiforme | GSE 4271 | Phillips (2006) | S4 | 76 | U133A |
| | GSE 8692 | Liu (2007) | S5 | 6 | U133A |
| | GSE 9171 | Wiedemeyer (2008) | S6 | 13 | U133 plus2.0 |
| | GSE 4290 | Sun (2006) | S7 | 77 | U133 plus2.0 |
| Medulloblastoma | GSE 10327 | Kool (2008) | S8 | 61 | U133 plus2.0 |
| | GSE 12992 | Fattet (2009) | S9 | 40 | U133 plus2.0 |
| Meningioma | GSE 4780 | Scheck (2006) | — | 62 | U133A/U133 plus2.0 |
| | GSE 9438 | Claus (2008) | S10 | 31 | U133 plus2.0 |
| | GSE 16581 | Lee (2010) | S11 | 66 | U133 plus2.0 |
| Oligodendrogiloma | GSE 4412 | Freije (2004) | S3 | 11 | U133A |
| | GSE 4290 | Sun (2006) | S7 | 50 | U133 plus2.0 |
| Pilocytic | GSE 12907 | Wong (2005) | S12 | 21 | U133A |
| Astrocytoma | GSE 5675 | Sharma (2007) | S13 | 41 | U133 plus2.0 |
| Normal Brain | GSE 3526 | Roth (2006) | S14 | 146 | U133 plus2.0 |
| | GSE 7307 | Roth (2007) | — | 57 | U133 plus2.0 |

*Studies that have not been published are denoted as ‘—’.

TABLE 4
Phenotype specimen descriptions and main results for all GEO accessions used

| Phenotype | GEO | First Author | | |
|---|---|---|---|---|
| Name | accession # | (publication year) | Phenotype specimen description | Main results |
| Ependymoma | GSE16155 | Donson (2009) | Human ependymoma tumor | Genes associated with nonrecurrent ependymoma were predominantly immune function-related |
| | | | resections | Histological analysis of a subset of immune function genes revealed that their expression |
| | | | | was restricted to tumor-infiltrating subpopulation |
| | | | | Up-regulation of immune function genes is the predominant ontology associated with a good prognosis in ependymoma |
| | GSE21687 | Johnson (2010) | Human ependymomas | Identified subgroups of ependymoma, and subgroup-specific gene amplifications and deletions |
| | | | comprised of minimum | Comparative transcriptomics between human tumors and mouse neural stem cells |
| | | | 85% tumour cells | generated mouse models of ependymoma with matching molecular expression patterns |
| | | | | Developed a novel cross-species genomic approach to match subgroup-specific |
| | | | | driver mutations with cellular compartments to model cancer subgroups |
| Glioblastoma | GSE4412 | Freije (2004) | Diffuse infiltrating gliomas | Gene expression-based grouping of tumors is a more powerful survival predictor than histologic grade or age |
| Multiforme | | | | The expression patterns of 44 genes classify gliomas into previously unrecognized biological and prognostic groups |
| | | | | Large-scale gene expression analysis and subset analysis of gliomas reveals unrecognized heterogenesity of tumors |
| | GSE4271 | Phillips (2006) | Primary high-grade | Novel prognostic subclasses of high-grade astrocytoma closely resemble stages in neurogenesis |
| | | | gliomas and matched recurrences | One tumor class displaying neuronal lineage markers shows longer survival, |
| | | | | while two tumor classes enriched for neural stem cell markers display equally short survival |
| | | | | Poor prognosis subclasses exhibit either markers of proliferation or of angiogenesis and mesenchyme |
| | | | | A robust two-gene prognostic model utilizing PTEN and DLL3 expression suggests that Akt and |
| | | | | Notch signaling are hallmarks of poor prognosis versus better prognosis gliomas, respectively |
| | GSE8692 | Liu (2007) | Primary low/high grade gliomas | Measured genome-wide mRNA expression levels and miRNA profiles by microarray analysis and RT-PCR, respectively |
| | | | | Correlation coefficients were determined for all possible mRNA-miRNA pairs |
| | | | | A subset of high correlated pairs were experimentally validated by overexpressing or suppressing |
| | | | | a miRNA and measuring the correlated mRNAs |
| | GSE9171 | Wiedemeyer | Glioblastoma tumors | A nonheuristic genome topography scan (GTS) algorithm was developed to characterize |
| | | (2008) | | the patterns of genomic alterations in human glioblastoma (GBM) |
| | | | | A codeletion pattern found among closely related INK genes in the GBM oncogenome challenges the prevailing single-hit |
| | | | | model of RB pathway inactivation |
| | | | | Results suggest a feedback regulatory circuit in the astrocytic lineage and demonstrate a bona fide |
| | | | | tumor suppressor role for p18 [symbol] in human GBM |
| | GSE4290 | Sun (2006) | Primary gliomas and | Stem cell factor (SCF) activates brain microvascular endothelial cells in vitro and |
| | | | nontumor brain samples | induces a potent angiogenic response in vivo |
| | | | | SCF downregulation inhibits tumor-mediated angiogenesis and glioma growth, whereas SCF overexpression |
| | | | | is associated with shorter survival in malignant glioma patients |
| | | | | The SCF/c-Kit pathway plays an important role in tumor- and normal host cell-induced angiogenesis within the brain |
| | | | | Anti-angiogenic strategies have great potential as a treatment approach for gliomas |
| Medulloblastoma | GSE10327 | Kool (2008) | Primary medulloblastomas | mRNA expression profiling and genomic hybridization arrays show 5 different types of medulloblastoma, |
| | | | and local relapses | each with characteristic pathway activation signatures and associated specific genetic defects |
| | | | | Clinicopathological features significantly different between the 5 subtypes include metastatic disease, |
| | | | | age at diagnosis, and histology |
| | GSE12992 | Fattet (2009) | Paediatric medulloblastomas | Immunostaining of β-catenin showed extensive nuclear staining in a subset of samples |
| | | | | Expression profiles show strong activation of the Wnt/ [symbol] -catenin pathway, and complete loss of chromosome 6 |
| | | | | Patients with extensive nuclear staining were significantly older at diagnosis and were in complete remission |
| | | | | after a mean follow-up of 75.7 months (range 27.5-121.2 months) from diagnosis |
| | | | | Results confirm previous observations that CTNNB1-mutated tumours represent a distinct molecular |
| | | | | subgroup of medulloblastomas with favourable outcome |
| Meningioma | GSE4760 | Scheck (2006) | Benign (grade 1) and aggressive | The results of this study have not been publicly disclosed |
| | | | (grades 2 and 3) meningiomas | |
| | GSE9438 | Claus (2008) | Meningioma specimens without | Progesterone and estrogen hormone receptors (PR and ER, respectively) were measured via |
| | | | neurofibromatosis type 2, | immunohistochemistry and compared with gene expression profiling results |
| | | | nonrecurrent | Gene expression seemed more strongly associated with PR status (+/−) than with ER status |
| | | | | Genes in collagen and extracellular matrix pathways were most differentially expressed by PR status |
| | | | | PR status may be a clinical marker for genetic subgroups of meningioma |
| Oligodendroglioma | GSE4412 | Philips (2004) | Primary high-grade gliomas | Novel prognostic subclasses of high-grade astrocytoma are identified and discovered to resemble stages in neurogenesis |
| | | | and matched recurrences | One tumor class displaying neuronal lineage markers shows longer survival, while two tumor classes |
| | | | | enriched for neural stem cell markers display equally short survival |
| | | | | Poor prognosis subclasses exhibit either markers of proliferation or of angiogenesis and mesenchyme |
| | | | | A roburst two-gene prognostic model utilizing PTEN and DLL3 expression suggests that Akt and Notch signaling are |
| | | | | hallmarks of poor prognosis versus better prognosis gliomas, respectively |
| | GSE4290 | Sun (2006) | Primary gliomas and nontumor | Stem cell factor (SCF) activates brain microvascular endothelial cells in vitro and induces |
| | | | brain samples | a program angiogenie response in vivo |
| | | | | Downregulation of SCF inhibits tumor-mediated angiogenesis and glioma growth in vivo, whereas overexpression of SCF is |
| | | | | associated with shorter survival in patients with malignant gliomas |
| | | | | The SCF/c-Kit pathway plays an important role in tumor- and normal host cell-induced angiogenesis within the brain |
| | | | | Antiangiogenic strategies have great potential as a treatment approach for gliomas |
| Pilocytic | GSE 12907 | Wong (2005) | Juvenile pilocytic astrocytomas | Genes involved in certain biological processes, including neurogenesis, cell adhesion, and central nervous |
| Astrocytoma | | | (JPAs) | system development, were significantly deregulated in JPA compared to those in normal cerebella |
| | | | | Two major subgroups of JPA based on unsupervised hierarchical clustering |
| | | | | JPA without myelin basic protein-positively stained tumor cells may have a higher tendency to progress |
| | GSE 5675 | Sharma (2007) | Primary pilocytic astrocytomas | No expression signature to discriminate clinically aggressive/recurrent tumors from indolent |
| | | | (PAs) arising sporadically and in | Unique gene expression pattern for PAs arising in patients with NF1 |
| | | | patients with neurofibromatosis | Gene expression signature stratified PAs by location (supratentorial versus infratentorial) |
| | | | type 1 (NF1) | Glial tumors may share an intrinsic, lineage-specific molecular signature that reflects the brain region |
| | | | | in which their nonmalignant predecessors originated |
| Normal Brain | GSE3526 | Roth (2006) | 20 anatomically distinct sites of | Principal component analysis and hierarchical clustering results showed that the expression |
| | | | the central nervous system (CNS) | patterns of the 20 CNS sites profiled were significantly different from all non-CNS |
| | | | 8 autopsies for each CNS region | tissues and were also similar to one another, indicating an underlying common expression signature |
| | | | Patient death was due to sudden death | The 20 sites could be segregated into discrete groups with underlying similarities in anatomical structure and, |
| | | | | in many cases, functional activity |
| | GSE7307 | Roth (2007) | Normal and diseased human tissues | The results of this study have not been publicly disclosed |
| | | | representing over 90 distinct | |
| | | | tissue types | |
| | | | Patient death was due to sudden death | |

[symbol]

indicates data missing or illegible when filed

Raw microarray intensity data (.CEL files) were obtained online from GEO and preprocessed simultaneously using identical techniques to reduce extraneous sources of technical artifacts. More specifically, common probe-sets were found across all transcriptome samples, and consensus preprocessing was performed on all the raw microarray image data to build a consensus dataset. This step removes one major non-biological source of variance between different studies. These preprocessed samples were used to build a multi-study, meta-dataset of human brain cancer and normal brain transcriptomes. Finally, stringent probe-set filtering was used to remove spurious classifier features.

The resulting hierarchical markers are shown above in Table 1. The discrimination at each node is shown in FIG. 1A.

A further summary is found in Table 5.

TABLE 5
Decision-Tree Marker-Panel Shows Phenotype-Specific
Signatures in the Form of Binary Patterns

| Gene symbols^{a} | | Phenotype binary signatures^{b} | | | | | | |
|---|---|---|---|---|---|---|---|---|
| Gene i | Gene j | EPN | GBM | MDL | MNG | OLG | PA | normal |
| PRPF40A | PURA | 1 | 1 | 1 | 1 | 1 | 1 | 0 |
| NRCAM | ISLR | 1 | 1 | 1 | 0 | 1 | 1 | — |
| SRI | NBEA | 1 | 1 | 0 | — | 1 | 1 | — |
| NUP62CL | OR10H3 | 1 | 0 | — | — | 0 | 0 | — |
| DDX27 | KCNMA1 | — | 1 | — | — | 1 | 0 | — |
| FLNA | TNKS2 | — | 1 | — | — | 0 | — | — |

In this table, the superscripts are as follows:
^{a}Affymetrix ® microarray platform probe IDs of the classifier genes are shown in Table 3 and Table 4.
^{b}For each gene-pair comparison (i.e., Is Gene i > Gene j ?), 1 and 0 delineates ‘true’ and ‘false’, respectively, and ‘—’ denotes that the outcome is not used for classification.

### EXAMPLE 2

### The Diagnostic Marker-Panel Achieves High Classification Accuracy in Cross-Validation

The classification performance of our brain cancer diagnostic marker-panel was first evaluated by ten-fold cross-validation. Our marker-panel achieved a 90.4% average of phenotype-specific classification accuracies (Table 6), showing strong promise for accurate diagnostics against a multi-category, multi-dataset background at the gene expression level.

TABLE 6
Classification Performance of Diagnostic Marker-Panel in Ten-Fold Cross-Validation

| | | Predicted phenotype (%)^{a} | | | | | | | | |
|---|---|---|---|---|---|---|---|---|---|---|
| | | EPN | GBM | MDL | MNG | OLG | PA | normal | UC^{b} | total |
| Actual phenotype | EPN | 92.2 | 2.8 | 0.3 | 1.7 | 1.3 | 0.6 | 0.2 | 1.0 | 102 |
| | GBM | 0.7 | 84.8 | 0.2 | 0.5 | 11.9 | 0.1 | 0.3 | 1.3 | 231 |
| | MDL | 2.2 | 2.3 | 91.1 | 0.8 | 2.7 | 0.2 | 0.0 | 0.8 | 101 |
| | MNG | 0.1 | 1.8 | 0.0 | 97.5 | 0.1 | 0.2 | 0.0 | 0.2 | 161 |
| | OLG | 0.5 | 20.7 | 0.2 | 0.0 | 74.6 | 2.1 | 0.0 | 2.0 | 61 |
| | PA | 1.3 | 2.3 | 0.0 | 0.0 | 1.3 | 94.4 | 0.0 | 0.8 | 62 |
| | normal | 0.0 | 0.5 | 0.0 | 0.1 | 0.7 | 0.0 | 98.5 | 0.1 | 203 |

In this table, the superscripts are as follows:
^{a}Accuracies reflect average performance in ten-fold cross-validation conducted ten times. The main diagonal gives the average classification accuracy of each class (bold), and the off-diagonal elements show the erroneous predictions.
^{b}UC (Unclassified samples). When using the node classifiers, expression profiles that did not exert a signature of any phenotype (i.e., did not percolate down to at least one positive terminal node) were rejected from classification. In this case, the Unclassified sample is treated as a misclassification.

In addition, we observed higher classification accuracy (93.2%) among the expression profiles for which a unique diagnosis was obtained without subsequent disambiguation from the decision-tree.

Four brain cancers (ependymoma, medulloblastoma, meningioma, and pilocytic astrocytoma) have accuracies of at least 91.1%, suggesting clear differences between them and the other phenotypes at the transcriptomics level. These cancers arise from unique cell types and regions in the brain, which affects the accuracy of the signatures. Ependymoma is composed of ependymal cells, which are the epithelial layer of the ventricular system of the brain and the spinal cord. Meningioma arises from the arachnoidal cells in the meninges, the system of membranes that covers and protects the central nervous system. Medulloblastoma is a neuroectodermal tumor derived from neural stem cell precursors originating in the cerebellum or posterior fossa. And finally, pilocytic astrocytoma is generally considered a low-grade, benign tumor of astrocytes, usually arising in the cerebellum or hypothalamus. Accordingly, the anatomical region specificity of these four cancers is likely to contribute toward their accurate separation—as there are regional areas of unique gene expression patterns, as discussed below.

The cross-validation accuracies for glioblastoma and oligodendroglioma, two well-progressed gliomas, were 84.8% and 74.6%, respectively. Their lower performance was mainly a consequence of the limited ability of the marker-panel to correctly differentiate these two cancers from each other. Indeed, the distinction of these two phenotypes seems to be rather difficult; although oligodendroglioma is generally characterized by its own unique histological features, it is also known to present morphological traits similar to those of glioblastoma. This suggests that the two phenotypes are not as clearly distinct as presently clinically defined. Interestingly, however, these two accuracies are comparable to those reported previously. Furthermore, our signatures did show an excellent degree of sensitivity (96.4%) and specificity (97.4%) for distinguishing these two well-progressed gliomas as a set from all other brain phenotypes. There exist genetic tests and methods that differentiate glioblastoma and oligodendroglioma well, such as the combined loss of chromosome arms 1p and 19q, and over-expression of transcription factors Olig1 and Olig2.

### EXAMPLE 3

### Use of Meta-Data

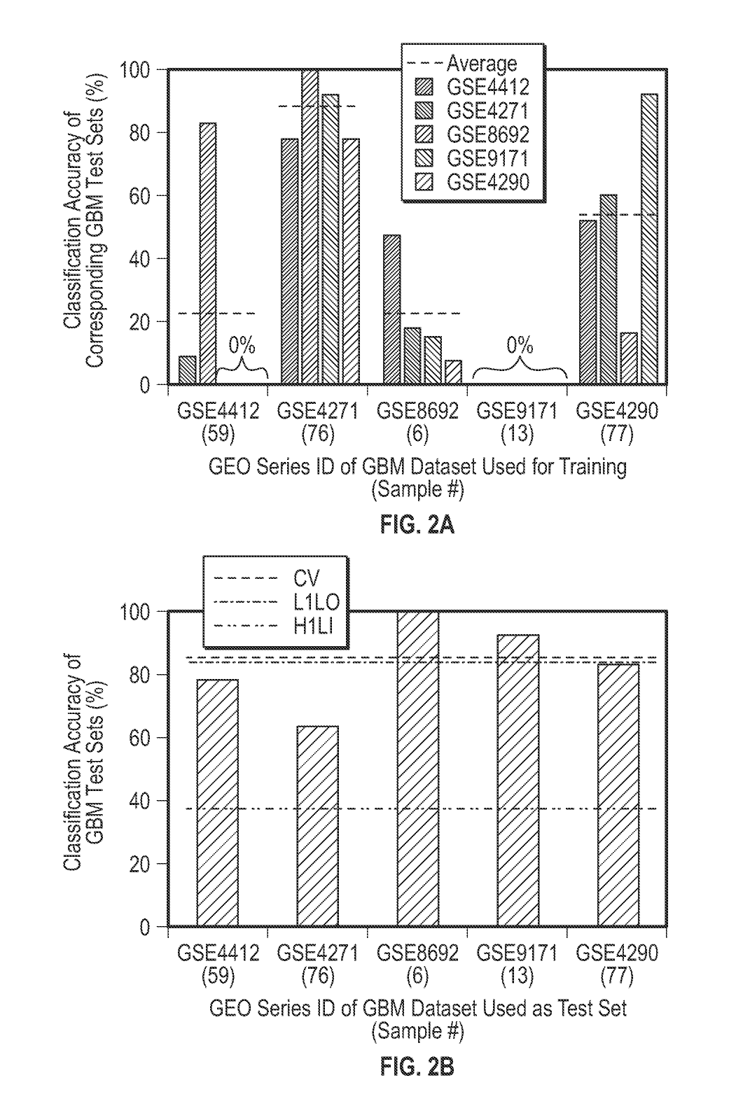

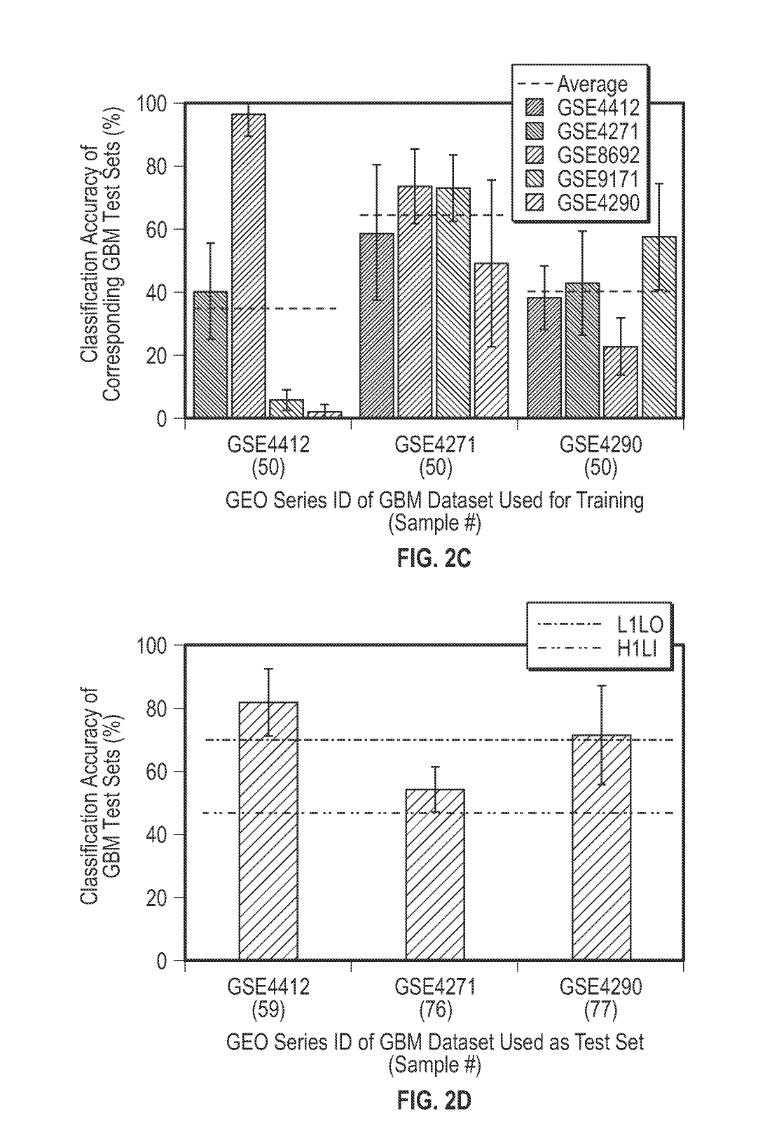

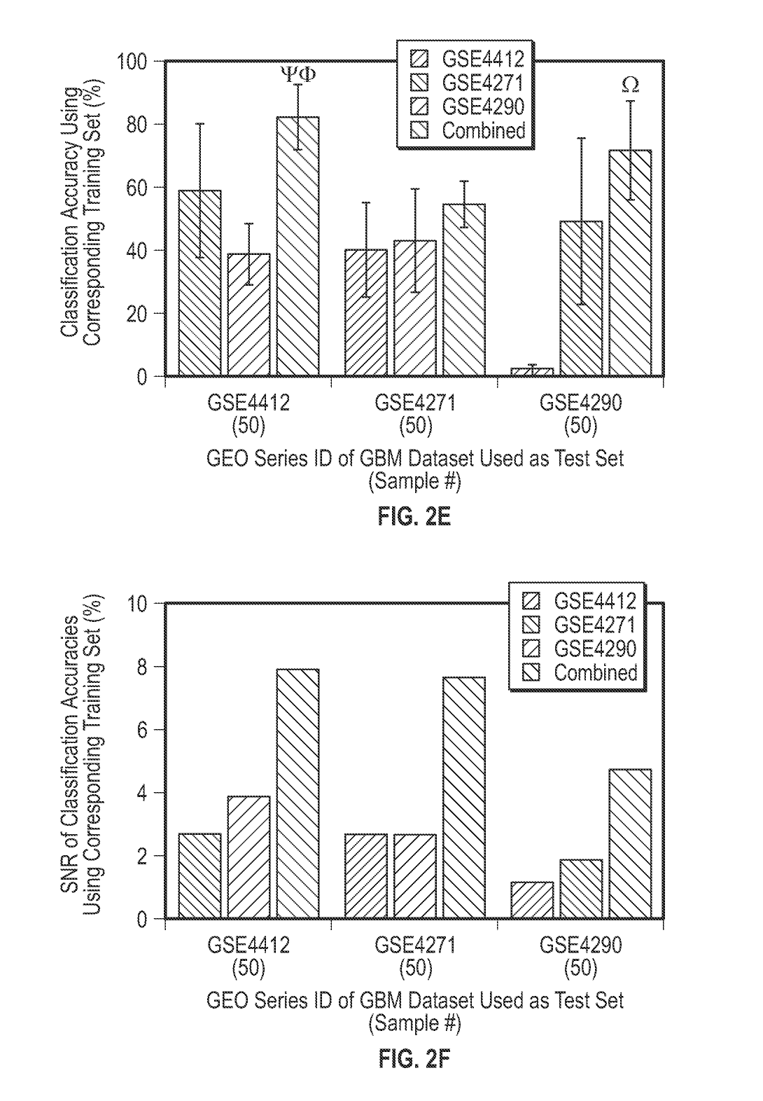

We trained ISSAC on each of the five transcriptomic datasets (i.e., GSE ####) of glioblastoma individually, coupled in each case to data from all other brain phenotypes. The results from various data handling methods are shown in FIGS. 2A-2F. The full multi-class signatures were completely relearned (every step) with the only difference in each case being which single glioblastoma dataset was included in the training stage. We then assessed the accuracy of correctly classifying glioblastoma transcriptomes measured in the four held-out datasets from all other possible phenotypes. We term this method of diagnostic signature evaluation as “hold-one-lab-in validation.” These are summarized in Table 7.

TABLE 7
Hold-one-lab-in validation accuracies of glioblastoma signatures.

| GBM training set | GBM test set | | | | | | | | |
|---|---|---|---|---|---|---|---|---|---|
| (sample size) | (sample size) | | Predicted phenotypes/% of test set/samples of test set | | | | | | |
| | | UC | EPN | GBM | MDL | MNG | OLG | PA | Total |
| GSE4412 (59) | GSE4271 (76) | 2.63% | 57.89% | 9.21% | 17.11% | 5.26% | 1.32% | 6.58% | 76 |
| | | 2 | 44 | 7 | 13 | 4 | 1 | 5 | 76 |
| | | GBM | MNG | Total | | | | | |
| | GSE8092 (0) | 83.33% | 16.67% | 6 | | | | | |
| | | 5 | 1 | 6 | | | | | |
| | | EPN | GBM | MNG | Total | | | | |
| | GSE9171 (13) | 92.31% | 0.00% | 7.00% | 13 | | | | |
| | | 12 | 0 | 1 | 13 | | | | |
| | | EPN | GBM | MDL | MNG | PA | Total | | |
| | GSE4290 (77) | 85.71% | 0.00% | 2.60% | 5.19% | 6.49% | 77 | | |
| | | 65 | 0 | 2 | 4 | 6 | 77 | | |
| | | UC | GBM | PA | normal | Total | | | |
| GSE4271 (76) | GSE 4412 (59) | 11.86% | 77.97% | 8.47% | 1.69% | 59 | | | |
| | | 7 | 46 | 5 | 1 | 59 | | | |
| | | GBM | Total | | | | | | |
| | GSE8692 (6) | 100.0% | 6 | | | | | | |
| | | 6 | 6 | | | | | | |
| | | GBM | 5 | Total | | | | | |
| | GSE9171 (13) | 92.31% | 7.69% | 13 | | | | | |
| | | 12 | 1 | 13 | | | | | |
| | | UC | GBM | MNG | PA | Total | | | |
| | GSE4290 (77) | 5.19% | 77.92% | 1.30% | 15.58% | 77 | | | |
| | | 4 | 60 | 1 | 12 | 77 | | | |
| | | UC | EPN | GBM | MDL | MNG | PA | normal | Total |
| GSE8092 (6) | GSE4412 (59) | 5.08% | 13.56% | 47.46% | 1.69% | 3.39% | 27.12% | 1.59% | 59 |
| | | 3 | 8 | 28 | 1 | 2 | 16 | 1 | 59 |
| | | UC | EPN | GBM | MDL | PA | normal | Total | |
| | GSE4271 (75) | 9.21% | 32.89% | 18.42% | 6.26% | 32.89% | 1.32% | 76 | |
| | | 7 | 25 | 14 | 4 | 25 | 1 | 76 | |
| | | EPN | GBM | MDL | PA | Total | | | |
| | GSE9171 (13) | 61.54% | 15.38% | 15.38% | 7.69% | 13 | | | |
| | | 8 | 2 | 2 | 1 | 13 | | | |
| | | UC | EPN | GBM | MDL | MNG | PA | normal | Total |
| | GSE4290 (77) | 14.29% | 42.86% | 7.79% | 1.30% | 1.30% | 26.97% | 6.49% | 77 |
| | | 11 | 33 | 6 | 1 | 1 | 20 | 5 | 77 |
| | | UC | EPN | GBM | MDL | MNG | PA | Total | |
| GSE9171 (13) | GSE4412 (59) | 35.59% | 13.56% | 0.00% | 1.69% | 5.08% | 44.07% | 59 | |
| | | 21 | 8 | 0 | 1 | 3 | 26 | 59 | |
| | | UC | EPN | GBM | MDL | MNG | PA | Total | |
| | GSE4271 (76) | 19.74% | 38.15% | 0.00% | 6.58% | 3.90% | 31.58% | 76 | |
| | | 15 | 29 | 0 | 5 | 3 | 24 | 76 | |
| | | UC | GBM | MNG | PA | Total | | | |
| | GSE8692 (6) | 66.67% | 0.00% | 16.67% | 16.67% | 6 | | | |
| | | 4 | 0 | 1 | 1 | 6 | | | |
| | | UC | EPN | GBM | MDL | PA | normal | Total | |
| | GSE4290 (77) | 10.39% | 40.26% | 0.00% | 1.30% | 46.75% | 1.30% | 77 | |
| | | 8 | 31 | 0 | 1 | 36 | 1 | 77 | |
| | | UC | GBM | NB | PA | normal | Total | | |
| GSE4290 (77) | GSE4412 (59) | 5.08% | 52.54% | 27.12% | 13.56% | 1.69% | 59 | | |
| | | 3 | 31 | 18 | 6 | 1 | 59 | | |
| | | UC | EPN | GBM | MDL | OLG | PA | Total | |
| | GSE4271 (76) | 1.32% | 1.32% | 60.53% | 3.95% | 15.79% | 17.11% | 76 | |
| | | 1 | 1 | 46 | 3 | 12 | 13 | 76 | |
| | | UC | GBM | NG | PA | Total | | | |
| | GSE8092 (6) | 33.33% | 16.67% | 16.67% | 33.33% | 6 | | | |
| | | 2 | 1 | 1 | 2 | 6 | | | |
| | | UC | GBM | Total | | | | | |
| | GSE9171 (13) | 7.69% | 92.31% | 13 | | | | | |
| | | 1 | 12 | 13 | | | | | |

In general, GBM signatures from larger datasets (GSE4271, GSE4290) had better average performance than those from smaller datasets (GSE8692, GSE9171), but variation across different validation sets limited overall performance (FIG. 2A). Training on GSE4271 (76 samples) resulted in the best overall average accuracy (87.1%) in correctly classifying samples from the four held-out glioblastoma datasets, with individual validation set accuracies ranging from 77.9% to 100% (Table 8).

TABLE 8
Ten-fold cross-validation accuracies when only the node marker-panel
was required to reach unique diagnoses.

| Phenotype | Total samples | Sample size (%) | Accuracy (%) |
|---|---|---|---|
| EPN | 102 | 93.1 | 95.8 |
| GBM | 231 | 88.9 | 92.7 |
| MDL | 101 | 95.0 | 95.8 |
| MNG | 161 | 98.8 | 97.5 |
| OLG | 61 | 77.0 | 74.5 |
| PA | 62 | 90.3 | 96.4 |
| Normal | 203 | 97.9 | 99.5 |
| Average | — | 91.6 | 93.2 |

Sample size: Average proportion of total samples that reached unique diagnoses via node marker-panel.
Accuracy: Reflects average performance in ten-fold cross-validation conducted ten times.

These favorable outcomes are likely due to the molecular heterogeneity within and across transcriptomes in this particular dataset adequately encompassing broad, population-level characteristics. This suggests that GSE4271 may serve as an ideal dataset in future studies for learning representative, molecular features of glioblastoma. Indeed, we found that training on GSE4271 was a notable exception; when GSE4290 (77 samples) was used as the training set, there was over a 30% decrease in average glioblastoma classification accuracy (55.5%), despite the nearly identical sample sizes of the two datasets. This shows that any individual dataset, even those of a sufficient sample size, do not consistently yield robust diagnostic signatures.

Signatures from GSE8692 (6 samples) and GSE9171 (13 samples), led to average accuracies of 22.3% and 0.0%, respectively; these significantly low performance results are not surprising given the very small sample numbers. However, that glioblastoma signatures from GSE9171 could not classify even a single sample correctly is an intriguing observation. After searching through sample preparation and handling protocols provided in the publications of all five glioblastoma studies, we were not able to identify any steps unique to the GSE9171 study that could have obviously led to such severe over-fitting. We suspect that, rather than from a single aspect, erroneous signals were obtained from a myriad of different factors, from the lack of variance in the biology of the patient samples studied, to batch effects that compromised transcriptomic measurements, and to possibly unreported variations in standard protocol. Finally, training on GSE4412 (59 samples) gave an average accuracy of 23.1%. Interestingly, the average accuracies from training sets GSE4412 and GSE8692 (23.1% and 22.3%, respectively) were very similar despite almost ten-fold difference in sample sizes (59 and 6 samples, respectively). This implies that, in general, sample size is really not a sole determining factor of signature performance. The overall hold-one-lab-in validation performance, or the average of all classification accuracies in FIG. 2A, was 37.6%.

We found considerable discrepancy between the minimum and maximum validation set accuracies for training sets GSE4412 (0.0% and 83.33%) and GSE4290 (16.7% and 92.31%) (Table 8). This shows that batch effects, as well as potential biological discrepancies between populations studied at different sites, can lead to remarkable variation among transcriptomic datasets of the supposedly same phenotype. This “dataset variation” is widespread in large-scale expression studies, causing inconsistencies in diagnostic signature identification and performance reproducibility. Large variation within and across transcriptomic datasets of glioblastoma is not surprising, given that glioblastoma is known to have various molecular subtypes. Therefore, as mentioned above, diagnostic signatures from any single dataset need to be approached with caution.

We next analyzed how the multi-study integration approach affects performance robustness. One of each of the five datasets of glioblastoma was sequentially withheld as the validation set, while all remaining gene expression data (including those from other phenotypes) were used for training. The glioblastoma signature was then evaluated on the held-out validation set. We term this strategy as “leave-one-lab-out validation.”

Classification accuracies ranged from 63.2% (GBM training set: 155 samples across four datasets; validation set: GSE4271, 76 samples) to 100% (GBM training set: 225 samples across four datasets; validation set: GSE8692, 6 samples) (FIG. 2B). The average accuracy of the five leave-one-lab-out validations was 83.3%, which is considerably higher than that obtained from training on individual glioblastoma datasets (37.6%), and is comparable to the glioblastoma accuracy seen in cross-validation (84.8%). Indeed, the fact that the glioblastoma classification accuracies from cross-validation and the leave-one-lab-out strategy are so close suggests that the effects of variability among the datasets from different institutions and time-points have been mostly overcome by integration across multiple training studies. We conjecture that this result is due to the underlying variation in the training sets better representing the true variation in the population, both by achieving a greater sample size, as well as by having the samples come from a broader range of situations.

To evaluate how multi-study dataset integration alone affects performance robustness, we performed hold-one-lab-in and leave-one-lab-out validations for GSE4412, GSE4271, and GSE4290 (59, 76, and 77 samples, respectively) while training on the same number of samples for glioblastoma. More specifically, the same steps in the analyses of FIG. 2A and FIG. 2B were used, while glioblastoma signatures were learned from a glioblastoma training set of 50 samples chosen randomly from either an individual dataset or across four combined datasets. This process was conducted ten times for each glioblastoma training set.

The results we observed from these analyses were consistent with our two aforementioned conclusions, as shown in Table 9.

TABLE 9
Hold-one-lab-in (H1LI) and leave-one-lab-out (L1LO) validation accuracies of glioblastoma
signatures when training data were constrained to 50 total samples.

| | GBM | | | | |
|---|---|---|---|---|---|
| | training set | | GBM prediction | | Average |
| Method | (50 samples) | GBM test set | Average accuracy | St. dev. | performance |
| H1LI | GSE4412 | GSE4271 | 40.26% | 14.98% | 36.39% |
| | | GSE8692 | 96.67% | 7.03% | |
| | | GSE9171 | 6.15% | 3.24% | |
| | | GSE4290 | 2.47% | 2.10% | |
| | GSE4271 | GSE4412 | 58.98% | 21.64% | 63.89% |
| | | GSE8692 | 74.00% | 11.43% | |
| | | GSE9171 | 73.08% | 10.41% | |
| | | GSE4290 | 49.46% | 26.56% | |
| | GSE4290 | GSE4412 | 38.47% | 10.23% | 40.66% |
| | | GSE4271 | 43.13% | 16.12% | |
| | | GSE8692 | 23.33% | 9.08% | |
| | | GSE9171 | 57.70% | 16.79% | |
| L1LO | GSE4271, GSE8692, | GSE4412 | 82.20% | 10.39% | 69.72% |
| | GSE9171, GSE4290 | | | | |
| | GSE4412, GSE8692, | GSE4271 | 54.87% | 7.18% | |
| | GSE9171, GSE4290 | | | | |
| | GSE4412, GSE4271, | GSE4290 | 72.08% | 15.29% | |
| | GSE8692, GSE9171 | | | | |

H1LI and L1LO validations were performed ten times for each category of training data. In each validation trial, 50 samples were randomly selected from the single microarray dataset (for H1LI) or from the multi-study, combined dataset (for L1LO).

First, when a diagnostic signature is learned from an individual dataset, its ability to accurately and precisely represent phenotype features across a broad population highly varies depending on the particular dataset used for training (FIG. 2C).

Second, combining datasets considerably increased average accuracy (FIG. 2D).

Thus, dataset integration across multiple studies, even without change in sample size, can lead to significant improvements in diagnostic performance.

We used the results in FIG. 2C and FIG. 2D to compare performances of different glioblastoma signatures on the same validation set (FIG. 2E). In all cases, glioblastoma signatures from combined datasets had, on average, higher classification accuracy than those from any of the individual datasets. These results were then used to evaluate the precision of a glioblastoma signature's classification accuracy by calculating its signal-to-noise ratio (SNR). SNR was calculated as the ratio of average classification accuracy to standard deviation. We found that, for all validation set cases, glioblastoma signatures developed on the basis of multiple datasets had SNRs greater by at least two fold than those from individual data sets. This clearly shows that learning on integrated, meta-datasets leads to diagnostic signatures that have higher and more consistent diagnostic performance (FIG. 2F).

When we performed the stringent test of obtaining a diagnostic signature from a single dataset of glioblastoma, we found the variation between individual studies often have a larger effect on the transcriptome than did phenotype differences, resulting in dramatically decreased average accuracy. However, we found that learning signatures across multiple datasets significantly improved average accuracy with concomitant reduction in performance variance, even when keeping the size of the training set the same. This was most likely due to the meta-signature encompassing more of the heterogeneity across different sources and conditions, while not losing focus on the important, global characteristics of the phenotype.

1. A reagent panel for distinguishing among samples that are normal and samples that harbor cancer wherein said cancer is selected from the group consisting of meningioma (MNG), ependymoma (EPN), medulloblastoma (MDL), glioblastoma (GBM), oligodendroglioma (OLG), and pilocytic astrocytoma (PA) or can distinguish samples that harbor one or more of said cancers from samples that harbor others of said cancers wherein said panel comprises pairs of detection reagents for the expression products of at least one selected gene pair among the following: PRPF40A and PURA; NRCAM and ISLR; IDH2 and GMDS; SALL1 and PAFAH1B3; SRI and NBEA; DDR1 and TIA1 or MAB21L1; ITPKB and PDS5B; NUP62CL and ZNF280A; GALNS and WAS; CELSR1 and OR10H3; TLE4 and OLIG2; DDX27 and KCNMA1; COX7A2 and GNPTAB; GNPTAB and NDUFS2; APOD and PPIA; CD59 and SNRPB2 or HINT1; SEMA3E and ADAMTS3; BAMBI and CIAPIN1; FLNA and TNKS2; ITGB3BP and RB1CC1; DDX27 and TRIM8; and LARP5 and ANXA1.

2. The reagent panel of claim 1 that comprises detection reagents for the expression products of the gene pair PRPF40A and PURA for distinguishing samples that are normal from samples that harbor cancer.

3. The reagent panel of claim 1 that comprises detection reagents for the expression products of the gene pairs NRCAM and ISLR and/or IDH2 and GMDS for distinguishing samples that harbor EPN, GBM, MDL, OLG or PA from samples that harbor MNG.

4. The reagent panel of claim 1 that comprises detection reagents for the expression products of the gene pairs SALL1 and PAFAH1B3; and/or SRI and NBEA; and/or DDR1^{e} and TIA1; and/or DDR1^{e} and MAB21L1; and/or ITPKB and PDS5B for distinguishing samples that harbor EPN, GBM, OLG or PA from samples that harbor MDL.

5. The reagent panel of claim 1 that comprises detection reagents for the expression products of the gene pairs NUP62CL and ZNF280A; and/or GALNS and WAS; and/or CELSR1 and OR10H3; and/or TLE4 and OLIG2 for distinguishing samples that harbor GBM, OLG or PA from samples that harbor EPN.

6. The reagent panel of claim 1 that comprises detection reagents for the expression products of the gene pairs KCNMA1 and DDX27; and/or GNPTAB and NDUFS2; and/or APOD and PPIA; and/or CD59 and SRNPB2; and/or SEMA3E and ADAMTS3; and/or CD59 and HINT1; and/or BAMBI and CIAPIN1 for distinguishing samples that harbor GMB or OLG from samples that harbor PA.

7. The reagent panel of claim 1 that comprises detection reagents for the expression products of the gene pairs LARP5 and ANXA1 for distinguishing samples that harbor GBM from samples that harbor OLG.

8. The reagent panel of claim 1 that comprises detection reagents for the expression products of at least two gene pairs.

9. The reagent panel of claim 1 that comprises detection reagents for the expression products of at least four gene pairs.

10. The reagent panel of claim 1 wherein said detection reagents detect mRNA.

11. A method to distinguish among normal samples, samples that harbor MNG, samples that harbor EPN, samples that harbor MDL, samples that harbor GBM, samples that harbor OLG, and samples that harbor PA which method comprises
initially distinguishing normal samples from samples that harbor any of the above-mentioned EPN, MDL, GBM, OLG and PA, followed by
distinguishing samples that harbor MNG from samples that harbor EPN, MDL, GBM, OLG or PA, followed by
distinguishing samples that harbor MDL from samples that harbor EPN, GBM, OLG or PA, followed by
distinguishing samples that harbor EPN from samples that harbor GBM, OLG or PA, followed by
distinguishing samples that harbor PA from samples that harbor GBM or OLG, followed by
distinguishing between samples that harbor GBM and samples that harbor OLG.

12. A method
(a) to distinguish samples that harbor cancer from normal samples which method comprises:
determining the level of expression of the PURA gene in said sample from a subject;
determining the level of expression of the PRPF40A gene in said sample;
comparing the level of expression of PURA and PRPF40A;
whereby a higher level of expression of PRPF40A as compared to PURA identifies the sample as harboring cancer and a lower level of expression of PRPF40A as compared to PURA identifies the sample as normal; or
(b) to distinguish samples that harbor meningioma (MNG) from samples that harbor alternative forms of cancer which method comprises:
determining the level of expression of the NRCAM gene in said sample;
determining the level of expression of the ISLR gene in said sample;
comparing the level of expression of NRCAM to the level of expression of ISLR; and/or
determining the level of expression of the IDH2 gene in said sample;
determining the level of expression of the GMDS gene in said sample;
comparing the level of expression of IDH2 to the level of expression of GMDS;
whereby a higher level of expression of ISLR as compared to NRCAM and/or a higher level of expression of GMDS as compared to IDH2 identifies the sample as harboring MNG; and
a lower level of expression of ISLR as compared to NRCAM and/or a lower level of expression of GMDS as compared to IDH2 identifies the sample as harboring an alternative form of cancer; or
(c) to distinguish samples that harbor medulloblastoma (MDL) from samples that harbor alternative forms of cancer which method comprises:
determining the level of expression of the PAFAH1B3 gene in a sample;
determining the level of expression of the SALL1 gene in said sample; and
comparing the level of expression of PAFAH1B3 and SALL1; and/or
determining the level of expression of the NBEA gene in said sample;
determining the level of expression of the SRI gene in said sample; and
comparing the level of expression of NBEA to the level of expression of SRI; and/or
determining the level of expression of the TIA1 gene or the MAB21L1 gene in said sample;
determining the level of expression of the DDR1 gene in said sample; and
comparing the level of expression of TIA1 or MAB21L1 to the level of expression of DDR1; and/or
determining the level of expression of the PDS5B gene in said sample;
determining the level of expression of the ITPKB gene in said sample;
comparing the level of expression of PDS5B with ITPKB;
whereby a higher level of expression of PAFAH1B3 as compared to SALL1; and/or
a higher level of expression of the NBEA gene as compared to the SRI gene; and/or
a higher level of the TIA1 gene or MAB21L1 gene as compared to DDR1; and/or
a higher level of the PDS5B gene as compared to ITPKB gene identifies the sample as harboring MDL; and
a lower level of expression of the PAFAH1B3 gene as compared to SALL1 gene; and/or
a lower level of expression of the NBEA gene as compared to SRI gene; and/or
a lower level of expression of the TIA1 gene or MAB21L1 gene as compared to DDR1; and/or
a lower level of expression of PDS5B as compared to ITPKB identifies the sample as harboring an alternative cancer; or
(d) A method to distinguish samples that harbor ependymoma (EPN) from samples that harbor alternative forms of cancer which method comprises:
determining the level of expression of the OLIG2 gene in a sample;
determining the level of expression of the TLE4 gene in said sample;
comparing the level of expression of OLIG2 to the level of expression of TLE4; and/or determining the level of expression of the WAS gene in said sample;
determining the level of expression of the GALNS gene in said sample;
comparing the level of expression of WAS to the level of expression of GALNS; and/or
determining the level of expression of the CELSR1 gene in said sample; and
determining the level of expression of the OR10H3 gene in said sample; and
comparing the level of expression of CELSR1 to the level of expression of OR10H3; and/or
determining the level of expression of the NUP62CL gene in said sample; and
determining the level of expression of the ZNF280A gene in said sample; and
comparing the level of expression of NUP62CL to the level of expression of ZNF280A;
whereby a higher level of expression of TLE4 as compared to the level of expression of OLIG2; and/or
a higher level of expression of GALNS as compared to the level of expression of WAS; and/or
a higher level of expression of CELSR1 as compared to the level of expression of OR10H3; and/or
a higher level of expression of NUP62CL as compared to the level of expression of ZNF280A identifies a sample as harboring EPN; and
whereby a lower level of expression of TLE4 as compared to the level of expression of OLIG2; and/or
a lower level of expression of GALNS as compared to the level of expression of WAS; and/or
a lower level of expression of CELSR1 as compared to the level of expression of OR10H3; and/or
a lower level of expression of NUP62CL as compared to the level of expression of ZNF280A identifies a sample as harboring an alternative form of cancer; or
(e) to distinguish samples that harbor PA from samples that harbor an alternative form of cancer, which method comprises
determining the level of expression of the KCNMA1 gene in a sample;
determining the level of expression of the DDX27 gene in said sample;
comparing the level of expression of KCNMA1 with that of DDX27; and/or
determining the level of expression of the GNPTAB gene in a sample;
determining the level of expression of the NDUFS1 gene in said sample; and
comparing the level of expression of GNPTAB and NDUFS1; and/or
determining the level of expression of the APOD gene in said sample;
determining the level of expression of the PPIA gene in said sample; and
comparing the level of expression of APOD to the level of expression of PPIA; and/or
determining the level of expression of the CD59 gene in said sample;
determining the level of expression of the SNRPB1 gene in said sample; and
comparing the level of expression of CD59 to the level of expression of SNRPB1; and/or
determining the level of expression of the SEMA3E gene in said sample;
determining the level of expression of the ADAMTS3 gene in said sample;
comparing the level of expression of SEMA3E with ADAMTS3; and/or
determining the level of expression of the CD59 gene in said sample;
determining the level of expression of HINT1 gene in a sample;
comparing the level of expression of CD59 to the level of expression of HINT1; and/or
determining the level of expression of the BAMBI gene in said sample;
determining the level of expression of the CIAPIN1 gene in said sample;
comparing the level of expression of BAMBI to the level of expression of CIAPIN1;
wherein a higher level of expression of KCNMA1 as compared to DDX27; and/or
a higher level of expression of GNPTAB as compared to NDUFS2; and/or
a higher level of expression of APOD as compared to PPIA; and/or
a higher level of expression of CD59 as compared to SNRPB2; and/or
a higher level of expression of SEMA3E as compared to ADAMT3; and/or
a higher level of expression of CD59 as compared to HINT1; and/or
a higher level of expression of BAMBI as compared to CIAPIN1 identifies the sample as harboring PA; and
a lower level of KCNMA1 as compared to DDX27; and/or
a lower level of expression of GNPTAB as compared to NDUFS2; and/or
a lower level of expression of APOD as compared to PPIA; and/or
a lower level of expression of CD59 as compared to SNRPB2; and/or
a lower level of expression of SEMA3E as compared to ADAMT3; and/or
a lower level of expression of CD59 as compared to HINT1; and/or
a lower level of expression of BAMBI as compared to CIAPIN1 identifies the sample as harboring an alternative form of cancer; or
(f) to distinguish samples that harbor GBM from samples that harbor an alternative form of cancer, which method comprises
determining the level of expression of the FLNA gene in a sample; and
determining the level of expression of the TNKS2 gene in said sample;
comparing the level of expression of FLNA with that of TNKS2; and/or
determining the level of expression of the ITGB3BP gene in a sample;
determining the level of expression of the RB1CC1 gene in said sample; and
comparing the level of expression of ITGB3BP and RB1CC1; and/or
determining the level of expression of the DDX27 gene in said sample;
determining the level of expression of the TRIM8 gene in said sample; and
comparing the level of expression of DDX27 to the level of expression of TRIM8;
wherein a higher level of expression of FLNA as compared to TNKS2; and/or
a higher level of expression of ITGB3P as compared to RB1CC1; and/or
a higher level of expression of DDX27 as compared to TRIM8 identifies the sample as harboring GBM; and
a lower level of expression of FLNA as compared to TNKS2; and/or
a lower level of expression of ITGB3P as compared to RB1CC1; and/or
a lower level of expression of DDX27 as compared to TRIM8 identifies the sample as harboring an alternative form of cancer; or
(g) to distinguish samples that harbor OLG from samples which harbor an alternative form of cancer which method comprises:
determining the level of expression of the ANXA1 gene in said sample;
determining the level of expression of the LARP5 gene in said sample; and
comparing the level of expression of ANXA1 and LARP5;
whereby a higher level of expression of LARP5 as compared to ANXA1 identifies the sample as harboring OLG and a lower level of expression of LARP5 as compared to ANXA1 identifies the sample as harboring an alternative form of cancer.

13.-18. (canceled)

19. The method of claims 11 or 12 wherein the sample is a sample of brain tissue or cerebral spinal fluid (CSF).

20. The method of claim 19 wherein the sample is brain tissue.

21. The method of claim 11 or 12 wherein the level of expression is determined by assessing messenger RNA.

22.-30. (canceled)
